(19) United States
(12) Patent Application Publication
(10) Pub. No.: US 2009/0253602 A1
(43) Pub. Date: Oct. 8, 2009

(54) Novel personal wash bar

(75) Inventors: Natasha Pfeiffer Campbell, Trumbull, CT (US); Shiping Zhu, Sharnbrook (GB)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/080,691

(22) Filed: Apr. 4, 2008

(51) Int. Cl. A61K 8/02; A61Q 19/10

(52) U.S. Cl. 510/146

(57) ABSTRACT

A solid moisturising personal wash bar with the characteristics of a moisturising cosmetic composition is desired. Previous attempts to provide such a solid moisturising personal wash bar have met with only partial success because of the limited amount of the moisturising cosmetic composition which may be incorporated into the solid personal wash bar before it becomes unsuitable for conventional soap bar manufacture as its hardness decreases and its stickiness increases.

The aforementioned drawback has been addressed by providing a solid moisturising personal wash bar comprising a continuous phase and a discontinuous phase, the discontinuous phase in the form of a plurality of discrete domains having a longest dimension of 100 to 20000 microns, wherein
the continuous phase is a solid personal wash composition comprising one or more surfactants,
the discontinuous phase is a solid anhydrous moisturising cosmetic composition comprising a first fatty acid material, a first organic base and an emollient and/or humectant, wherein
   the degree of neutralisation of the first fatty acid material is 8-60%, preferably 10-50%, most preferably 15-35%.

This invention relates to a novel solid personal wash bar and manufacture thereof. In particular the invention relates to a solid moisturising personal wash bar comprising a solid anhydrous moisturising cosmetic composition.

In the context of the present invention, the term “solid” preferably refers to a non-sticky hard or soft solid material that does not flow or does not visibly deform when observed after being filled as a melt into a 250 ml glass laboratory beaker (with an height:diameter ratio of 2:1) to a level which is half of the internal height of beaker, then cooled to and maintained at 25° C., the beaker then being fixed at a tilt angle 45° to the horizontal, the observation being made 5 minutes after moment of first tilting.

Solid personal wash bars are effective in removing dirt and oil but have a tendency to dry the skin leading to skin irritation and flaking. These drawbacks can be ameliorated, to some degree, by homogeneously incorporating an emollient, such as petroleum jelly, and/or a humectant, such as glycerine, into the solid personal wash bar. However only a limited amount of the emollient and/or humectant may be incorporated into the solid personal wash bar before it becomes unsuitable for conventional soap bar manufacture as its hardness decreases and its stickiness increases.

An alternative route to incorporating an emollient and/or humectant into a solid personal wash bar is through homogeneous incorporation of a moisturising cosmetic composition comprising a first fatty acid material, a first organic base and an emollient and/or humectant wherein the degree of neutralisation of the first fatty acid material is 8-60%, preferably 10-50%, most preferably 15-35%. This route has the advantage of combining the characteristics of a personal wash composition with those of a moisturising cosmetic composition and not merely an emollient and/or humectant. However as the moisturising cosmetic composition typically comprises 60-90% by weight of the moisturising cosmetic composition of water, this alternative route suffers from the same problem set forth hereinabove in that only a limited amount of the moisturising cosmetic composition may be incorporated into the solid personal wash bar before it becomes unsuitable for conventional soap bar manufacture as its hardness decreases and its stickiness increases.

Thus there remains a continuing and unmet need for the provision of a solid personal wash bar which addresses the aforementioned drawbacks of existing solid personal wash bars.

### SUMMARY OF THE INVENTION

In a first aspect of the invention, a solid moisturising personal wash bar is provided comprising a continuous phase and a discontinuous phase, the discontinuous phase in the form of a plurality of discrete domains having a longest dimension of 100 to 20000 microns, wherein
the continuous phase is a solid personal wash composition comprising one or more surfactants,
the discontinuous phase is a solid anhydrous moisturising cosmetic composition comprising a first fatty acid material, a first organic base and an emollient and/or humectant, wherein
   the degree of neutralisation of the first fatty acid material is 8-60%, preferably 10-50%, most preferably 15-35%

Surprisingly it has been observed by the inventors that by incorporating a solid anhydrous moisturising cosmetic composition comprising a first fatty acid material, a first organic base and an emollient and/or humectant wherein the degree of neutralisation of the first fatty acid material is 8-60%, preferably 10-50%, most preferably 15-35% into a solid personal wash bar in the form of a plurality of discrete domains having a longest dimension of 100 to 20000 microns rather than homogeneously, a solid personal wash bar is provided which combines the characteristics of a personal wash composition with those of a moisturising cosmetic composition, the latter hydrating on contact with water in the temperature range of 10° C. to 80° C., without the formulation limitations set forth hereinabove.

The first fatty acid material may include blends of fatty acids, typically containing fatty acid moieties with chain lengths of from C₁₄ to C₃₀. The first fatty acid material may also contain relatively pure amounts of one chain length fatty acid moiety. Suitable fatty acids include lauric, myristic, palmitic, stearic, isostearic, oleic, linoleic, ricinoleic, arachidic, behenic and erucic acids. In certain preferred embodiments, the fatty acid moiety chain length is from 14 to 22, preferably 14 to 20, more preferably 16 to 18 carbon atoms.

Although normally saturated, suitable first fatty acid material may comprise unsaturated fatty acid moieties, and may comprise fatty acid moieties having a degree of substitution, such as e.g. hydroxy fatty acids. Preferred first fatty acid material is selected from the group comprising saturated fatty acids or a combination of saturated fatty acids and unsaturated fatty acids. The melting points of the individual fatty acids are relatively unimportant insofar as what is important is the overall melting point of the first fatty acid material, which should be in the range 41° C. to 80° C.

The first fatty acid material may comprise relatively high amounts (e.g. at least 50%, preferably at least 75%) of stearic and/or palmitic acid moieties. The chain length of the fatty acid material used according to the invention determines the rheological properties of the resultant moisturising cosmetic composition. A first fatty acid material comprising relatively high proportions of stearic and palmitic acid moieties has been found to be particularly suitable for use for manufacturing moisturising cosmetic compositions which may be used in temperate to hot climates as, when stearic and/or palmitic acid is partially neutralised with an organic base, a desirable lamellar gel (beta) phase is formed which is relatively viscous.

The degree of neutralisation of the first fatty acid material is selected so that it is not so low that the anhydrous moisturising cosmetic composition will not hydrate in water and not so high as to raise the pH of the moisturising cosmetic composition much beyond that of skin (which is pH 5.5) which would lead to skin irritation. In the latter case, the moisturising cosmetic composition would also feel slimey. The moisturising cosmetic composition would typically have a pH in the range 6 to 9, preferably 6.5 to 8.

The temperature at which a surfactant will solubilise in a solvent is known as the Krafft point. More specifically the Krafft point of a surfactant is defined as the temperature at which the solubility of the surfactant increases significantly to reach the critical micelle concentration. Below this point, there is very little solubility. The Krafft point can be determined by locating the abrupt change in slope of a graph of the logarithm of the solubility against the reciprocal of the temperature. Thus the Krafft point of the partially neutralised first fatty acid material is at or below the temperature of the water which hydrates the anhydrous moisturising cosmetic composition.

A first organic base is essential so that the partially neutralised first fatty acid material has a Krafft point in the temperature range of 10° C. to 80° C. Partial neutralisation by an inorganic base would render the first fatty acid material comprising more than 50% w/w palmitic and stearic acids insoluble at temperatures below 60° C. When in use in water below 60° C., such a solid personal wash bar would feel gritty (from insoluble partially neutralised C₁₆ and C₁₈ fatty acids) if the discontinuous phase is in the form of discrete domain having a longest dimension of 100-10000 microns. Furthermore the solid personal wash bar would not be effective in moisturising the skin as a proportion of the emollient and/or humectant would remain trapped in the insoluble portion of the anhydrous moisturising cosmetic composition.

The first organic base may be selected from the group consisting of monoethanolamine, triethanolamine, triisopropanolamine and mixtures thereof.

The discrete domains may have a longest dimension of 200 to 10000, preferably 250 to 5000, most preferably 300 to 3000 microns. Preferably at least 50%, more preferably at least 70%, most preferably at least 90% by weight of the solid anhydrous cosmetic composition is in the form of discrete domains having a longest dimension of 100 to 20000, preferably 200 to 10000, more preferably 250 to 5000, most preferably 300 to 3000 microns.

The emollient and/or humectant may be selected from the group consisting of petroleum jelly, glycerine, mineral oil, vegetable oils and mixtures thereof; preferably petroleum jelly or glycerine.

The emollient material may also be a silicone oil, an ester or a mixture thereof.

Silicone oils may be divided into those which are volatile and those which are not. The term “volatile” as used herein refers to those materials which have a measurable vapour pressure at ambient temperature. Volatile silicone oils are preferably chosen from cyclic or linear polydimethyl siloxanes containing from 3 to 9, preferably from 4 to 5 silicon atoms. Linear volatile silicone materials generally have viscosities less than 5 centistokes (5×10⁻⁶ m²/s) at 25° C., whilst cyclic materials typically have viscosities of less than 10 centistokes (10⁻⁵ m²/s) at 25° C.

Non-volatile silicone oils useful as an emollient material include polyalkyl siloxanes, polyalkylaryl siloxanes and polyether siloxane copolymers. The non-volatile polyalkyl siloxanes useful herein include, for example, polydimethyl siloxanes with viscosities of from 5-25×10⁶ centistokes (5×10⁻⁶-25 m²/s) at 25° C. Among the preferred non-volatile silicone emollients useful in the present compositions are the polydimethyl siloxanes having viscosities from about 10-400 centistokes (10⁻⁵-4×10⁻⁴ m²/s) at 25° C.

Among the ester emollients are:
(1) Alkenyl or alkyl esters of fatty acids having 10 to 20 carbon atoms. Examples thereof include isoarachidyl neopentanoate, isononyl isononanoate, oleyl myristate, oleyl stearate and oleyl oleate.
(2) Ether-esters such as fatty acid esters of ethoxylated fatty alcohols.
(3) Polyhydric alcohol esters. Ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate and arachidyl behenate.
(5) Sterol esters of which cholesterol fatty acid esters are examples thereof.

Other humectants of the polyhydric alcohol type may also be employed. The humectant also aids in increasing the effectiveness of the emollient, reduces scaling, stimulates removal of built-up scale and improves skin feel. Typical polyhydric alcohols include polyalkylene glycols (such as polypropylene glycol, polyethylene glycol and dipropylene glycol), alkylene polyols (such as propylene glycol), sorbitol, hydroxypropyl sorbitol, hexylene glycol, 1,3-butylene glycol, 1,2,6-hexanetriol, ethoxylated glycerol, propoxylated glycerol, derivatives thereof and mixtures thereof.

The amount of first fatty acid material is preferably 70-95, more preferably 80-95% by weight of the solid anhydrous moisturising cosmetic composition.

The amount of the emollient and/or humectant is preferably 0.1-25, most preferably 1-20% by weight of the solid anhydrous moisturising cosmetic composition.

The solid moisturising personal wash bar may comprise 60-98, preferably 70-95, most preferably 75-95% by weight of the solid moistunsing personal wash bar of solid personal wash composition.

The solid personal wash composition preferably comprises:

(a) a second fatty acid material;

(b) a second organic base or an inorganic base or a mixture thereof; and

(c) 10-20% by weight of the solid personal wash composition of water; wherein

the second fatty acid material is at least partially neutralised with the second organic base or the inorganic base or the mixture thereof, and wherein
the degree of neutralisation of the second fatty acid material is at least 80%, preferably at least 90%, most preferably at least 95%.

The second fatty acid material may include blends of fatty acids, typically containing fatty acid moieties with chain lengths of from C₈ to C₃₀. The second fatty acid material may also contain relatively pure amounts of one chain length fatty acid moiety. Suitable fatty acids include pelargonic, lauric (C₁₂), myristic (C₁₄), palmitic (C₁₆), stearic (C₁₈), isostearic, oleic, linoleic, ricinoleic, arachidic, behenic and erucic acids. Although normally saturated, suitable second fatty acid material may contain unsaturated fatty acid moieties, and may contain fatty acid moieties having a degree of substitution, such as e.g. hydroxy fatty acids.

The second fatty acid material comprises relatively high amounts of lower chain length fatty acid moieties (e.g. more than 50% of the fatty acid moiety having a chain length of C₈-C₁₄) because, when partially neutralised with an organic and/or inorganic base, these lower chain length fatty acid moieties, in the presence of water, are already in the lamellar liquid (alpha) phase at 25° C. and able to foam. Foams have been observed to lead to more effective detergency in solid personal wash bars. In the absence of water, the partially neutralised second fatty acid material would not foam very readily.

In contrast, partially neutralised higher chain length fatty acid moieties (e.g. fatty acid moieties having a chain length of C₁₆-C₃₀) are in the form of crystals and provide structure to the solid personal wash composition.

The second organic base may be selected from the group consisting of monoethanolamine, triethanolamine, triisopropanolamine or a mixture thereof. The first and second organic bases may be the same. The inorganic base may be selected from the group consisting of sodium hydroxide, potassium hydroxide or a mixture thereof.

The solid personal wash composition may comprise at least 80, preferably at least 90% by weight of the solid moisturising personal wash composition of the second fatty acid material.

The foregoing embodiment of solid personal wash composition may be produced by the well-known kettle boiling process or modern continuous soap manufacturing processes wherein natural fats and oils such as tallow or coconut oil or their equivalents are saponified with an alkali metal hydroxide, or other base, using procedures well known to those skilled in the art. Alternatively, the soaps may be made by neutralizing fatty acids, such as lauric (C₁₂), myristic (C₁₄), palmitic (C₁₆), or stearic (C₁₈) acids with an alkali metal hydroxide or carbonate, or other base.

### DETAILED DESCRIPTION OF THE INVENTION

The solid anhydrous moisturising cosmetic composition can also comprise optional ingredients such as other surfactants, colorant, fragrance, thickeners and sunscreens.

Suitable other surfactants can be selected from the group consisting of fatty alcohols, fatty acid esters and a mixture thereof. Cetyl alcohol is an example of a fatty alcohol, and glycerol stearate and glycol stearate are examples of fatty acid esters.

Suitable thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. A further alternative thickener is clay. Amounts of the thickener, if at all, are 0.0001-2, preferably 0.001-1% by weight of the solid anhydrous moisturising cosmetic composition.

Suitable sunscreens include azobenzophenone (Parsol 1789®)), octyl methoxycinnamate (Parsol MCX) and 2-hydroxy-4-methoxy benzophenone (also known as oxybenzone and sold under the trade name of Benzophenone-3). The exact amount of sunscreen employed in the solid anhydrous moisturising cosmetic composition varies depending upon the degree of ultra-violet radiation protection desired.

The solid personal wash composition may also comprise synthetic surfactants which are not based on neutralisation of fatty acids amongst anionic, cationic, non-ionic, zwitterionic and amphoteric surfactants.

Suitable anionic surfactants may include, for example, an aliphatic sulphonate, an alkyl sulphate, an alkyl ether sulphate, an alkyl sulphosuccinate, an alkyl taurate, an acyl taurate, an alkyl sarcosinate, an acyl sarcosinate, a sulphoacetate, a C₈-C₂₂ alkyl phosphate, an alkyl phosphate ester, an alkoxyl alkyl phosphate ester, an acyl lactate, a C₈-C₂₂ monoalkyl succinate, a maleate, a sulphoacetate, an acyl isethionate, an alkoxy carboxylate, an amido alkyl polypeptide carboxylate, a C₈-C₁₈ acyl isethionate and an acyl alkoxy isethionate.

Suitable cationic surfactants are quaternary ammonium compounds such as alkyldimethylammonium halogenides.

Suitable non-ionic surfactants may include, for example, an alkoxy aliphatic alcohol, an alkoxy aliphatic acid, an alkoxy aliphatic amide, an alkoxy alkyl phenol, a long chain tertiary amine oxide, a long chain tertiary phosphine oxide, a dialkyl sulphoxide, a sugar amide and an alkyl polysaccharide.

Suitable zwitterionic surfactants may include, for example, an aliphatic quaternary ammonium compound, an aliphatic phosphonium compound and an aliphatic sulphonium compound. Examples of such zwitterionic surfactants include 4-[N,N-di(2-hydroxyethyl)-N-octadecylammonio]-butane-1-carboxylate, 5-[S-3-hydroxypropyl-S-hexadecylsulphonio]-3-hydroxypentane-1-sulphate, 3-[P,P-diethyl-P-3,6,9-trioxatetradexocylphosphonio]-2-hydroxypropane-1-phosphate, 3-[N,N-dipropyl-N-3-dodecoxy-2-hydroxypropylammonio]-propane-1-phosphonate, 3-(N,N-dimethyl-N-hexadecylammonio)propane-1-sulphonate, 3-(N,N-dimethyl-N-hexadecylammonio)-2-hydroxypropane-1-sulphonate, 4-[N,N-di(2-hydroxyethyl)-N-(2-hydroxydodecyl)ammonio]-butane-1-carboxylate, 3-[S-ethyl-S-(3-dodecoxy-2-hydroxypropyl)sulphonio]-propane-1-phosphate; 3-[P,P-dimethyl-P-dodecylphosphonio]-propane-1-phosphonate and 5-[N,N-di(3-hydroxypropyl)-N-hexadecylammonio]-2-hydroxy-pentane-1-sulphate.

Suitable amphoteric surfactants may include, for example, a betaine, a sulphobetaine, an amphoacetate and a diamphoacetate.

In general the solid personal wash composition may comprise 1-20%, preferably 2-15%, most preferably 5-12% by weight of the solid personal wash composition of the aforementioned suitable anionic, cationic, non-ionic, zwitterionic, amphoteric surfactants or mixtures thereof.

The solid personal wash composition may also comprise further optional ingredients selected from the group consisting of chelating agents, preservatives, antioxidants, perfumes, skin benefit agents and optical modifiers.

An example of a suitable chelating agent is ethylene diamine tetra acetic acid (EDTA).

Examples of suitable preservatives include dimethylol dimethyl hydantoin (Glydant™ XL1000), methyl parabens, propyl parabens and sorbic acid.

An example of a suitable antioxidant is butylated hydroxytoluene (BHT).

Examples of suitable skin benefit agents include antimicrobial agents, anti-acne agents, oil control agents, mattifiers, astringents, scrub/exfolliating particles, cooling agents, fruit/herbal extracts, skin calming agents and essential oils.

Specific suitable examples of antimicrobial agents include 2-hydroxy-4,2′,4′-trichlorodiphenylether (DP300), 2,6-dimethyl-4-hydroxychlorobenzene (PCMX), 3,4,4′-trichlorocarbanilide (TCC), 3-trifluoromethyl-4,4′-dichlorocarbanilide (TFC), benzoyl peroxide, zinc salts and tea tree oil.

Specific suitable examples of anti-acne agents include salicylic acid, lactic acid, glycolic acid, citric acid and benzoyl peroxide.

Specific suitable examples of a mattifier include silica, titanium dioxide and microsponges.

Specific suitable examples of an astringent include tannins, zinc and aluminium salts, green tea extract and Witchhazel (Hamamelis) extract.

Specific suitable examples of scrub/exfolliating particles include polyethylene spheres, agglomerated silica, sugar, salts, and ground pits, seeds, and husks from walnut, peach, avocado and oats.

Specific suitable examples of cooling agents include menthol and its various derivatives, and lower alcohols.

An example of a suitable skin calming agent is aloe vera.

Specific suitable examples of an essential oil include mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, menthol, cineole, eugenol, citral, citronelle, borneol, linalool, geranoil, evening primrose, camphor, thymol, spirantol, penene, limonene and terpenoid oils.

Examples of suitable optical modifiers include ground fruit pits, seeds, polyethylene beads, mineral agglomerates, loofah, mica, coated micas and titanium oxide particles.

The bars according to the invention may be prepared in at least two steps. In the first step the solid anhydrous moisturising cosmetic composition is prepared by mixing the first fatty acid material with the emollient and/or humectant and heating the mixture to form a melt. The first organic base is then added to the melt under stirring to form an isotropic liquid. The isotropic liquid is then cooled to room temperature. The resulting solid is then ground and sieved to a selected particle size range.

In the second step the solid personal wash composition and the sieved solid anhydrous moisturising cosmetic composition are mixed together at room temperature and the resulting mixture extruded through a die at a temperature not exceeding 40° C. to form a billet without further mixing steps. The billet may then be cut and stamped to form a finished bar.

### EXAMPLE

Solid moisturising personal wash bars were prepared with 20% w/w solid anhydrous moisturising cosmetic composition both as homogenous bars and according to the invention. The solid anhydrous moisturising cosmetic composition comprised either 10% or 20% by weight of the solid anhydrous moisturising cosmetic composition of petrolatum G1937 (Crompton Corporation) in accordance with the formulations hereinbelow.

### Solid Anhydrous Moisturising Cosmetic Composition Formulation 1 (10% w/w Petrolatum G1937)

| Ingredient | Weight (%) |
|---|---|
| Pristerene 4911 (a) | 74.99 |
| Petrolatum G1937 | 10.00 |
| Triethanolamine (91%) (b) | 15.01 |

(a) Mixture of palmitic and stearic acids; available from Unichema
(b) In water

### Solid Anhydrous Moisturising Cosmetic Composition Formulation 2 (20% w/w Petrolatum G1937)

| Ingredient | Weight (%) |
|---|---|
| Pristerene 4911 (a) | 66.66 |
| Petrolatum G1937 | 20.00 |
| Triethanolamine (91%) (b) | 13.34 |

(a) Mixture of palmitic and stearic acids; available from Unichema
(b) In water

The two formulations of solid anhydrous moisturising cosmetic composition were prepared by mixing Pristerene 4911 with petrolatum G1937 and heating the mixture to 80° C. to form a melt. The triethanolamine was then added to the melt under stirring to form an isotropic liquid. The isotropic liquid was then cooled to room temperature. The resulting solid was then ground and sieved to a selected particle size range of 2.8-20 millimetres.

The solid personal wash composition formulation is set forth hereinbelow.

### Solid Personal Wash Composition Formulation

| Ingredient | Weight (%) |
|---|---|
| 80/20 tallow/palm kernel oil soap | 100.00 |

The 80/20 tallow/palm kernel oil soap was purchased in the form of short noodles (10-30 millimetres in length and 6-8 millimetres in diameter).

The homogeneous bars were prepared by adding the aforementioned solid anhydrous moisturising cosmetic composition in the form of a melt (obtained by heating the solid anhydrous moisturising cosmetic composition to 80° C.) to the 80/20 tallow/palm kernel oil soap at room temperature. The resultant mixture was then mixed further by passing it through an extruder, then a rolling mill and then another extruder. The product of these mixing steps was then extruded through a rectangular die at 40° C., cut and stamped into homogeneous bars.

The bars according to the invention were prepared by mixing together the 80/20 tallow/palm kernel oil soap and the sieved solid anhydrous moisturising cosmetic composition at room temperature and extruding the resulting mixture through a rectangular die at 40° C. to form a rectangular billet without the further mixing steps required for the preparation of the homogeneous bars as set forth hereinabove. The rectangular billet was then cut and stamped to form a finished bar.

The results were that bars according to the invention were produced with both solid anhydrous moisturising cosmetic composition formulations but an attempt to produce a homogeneous bar with solid anhydrous moisturising cosmetic composition formulation 2 was unsuccessful because the homogeneous mixture of 80/20 tallow/palm kernel oil soap and the sieved solid anhydrous moisturising cosmetic composition proved too soft and sticky to be passed through the soap extruder and/or cut and stamped.

1. A solid moisturising personal wash bar comprising a continuous phase and a discontinuous phase, the discontinuous phase in the form of a plurality of discrete domains having a longest dimension of 100 to 20000 microns, wherein
the continuous phase is a solid personal wash composition comprising one or more surfactants,
the discontinuous phase is a solid anhydrous moisturising cosmetic composition comprising a first fatty acid material, a first organic base and an emollient and/or humectant, wherein
the degree of neutralisation of the first fatty acid material is 8-60%, preferably 10-50%, most preferably 15-35%.

2. A solid moisturising personal wash bar according to claim 1 wherein the discrete domains have a longest dimension of 200 to 10000, preferably 250 to 5000, most preferably 300 to 3000 microns.

3. A solid moisturising personal wash bar according to claim 1 wherein the emollient and/or humectant is selected from the group consisting of petroleum jelly, glycerine, mineral oil, vegetable oils and mixtures thereof; preferably petroleum jelly or glycerine.

4. A solid moisturising personal wash bar according to claim 1 wherein the solid anhydrous moisturising cosmetic composition comprises 70-95, preferably 80-95% by weight of the solid anhydrous moisturising cosmetic composition of the first fatty acid material.

5. A solid moisturising personal wash bar according to claim 1 wherein the solid anhydrous moisturising cosmetic composition comprises 0.1-25, preferably 1-20% by weight of the solid anhydrous moisturising cosmetic composition of the emollient and/or humectant.

6. A solid moisturising personal wash bar according to claim 1 comprising 60-98, preferably 70-95, most preferably 75-95% by weight of the solid moisturising personal wash bar of solid personal wash composition.

7. A solid moisturising personal wash bar according to claim 1 wherein the solid personal wash composition comprises:
a) a second fatty acid material;
b) a second organic base or an inorganic base or a mixture thereof; and
c) 10-20% of the solid personal wash composition of water; wherein
the second fatty acid material is at least partially neutralised with the second organic base or the inorganic base or the mixture thereof, and wherein.
the degree of neutralisation of the second fatty acid material is at least 80%, preferably at least 90%; most preferably at least 95%.

8. A solid moisturising personal wash bar according to claim 7 wherein the solid personal wash composition comprises at least 80, preferably at least 90% by weight of the solid moisturising personal wash composition of the second fatty acid material.
